(19) United States
(12) Patent Application Publication
(10) Pub. No.: US 2023/0285065 A1
(43) Pub. Date: Sep. 14, 2023

(54) ELECTROSURGICAL INSTRUMENTS INCLUDING THERMAL CUTTING ELEMENTS

(71) Applicant: Covidien LP, Mansfield, MA (US)

(72) Inventors: James D. Allen, IV, Broomfield, CO (US); William E. Robinson, Boulder, CO (US); Daniel A. Joseph, Golden, CO (US); John A. Hammerland, III, Arvada, CO (US); Kenneth E. Netzel, Loveland, CO (US)

(21) Appl. No.: 18/019,945

(22) PCT Filed: Aug. 11, 2021
(86) PCT No.: PCT/US2021/045540

§ 371 Date: Feb. 6, 2023

Related U.S. Application Data
(60) Provisional application No. 63/072,840, filed on Aug. 31, 2020.

(51) Int. Cl. A61B 18/08

(52) U.S. Cl. CPC A61B 18/085; A61B 2018/00601

(57) ABSTRACT

An electrosurgical instrument including an end effector assembly having first and second jaw members and at least first and second thermal cutting elements. Each of the first and second jaw members includes an electrically conductive tissue contacting surface. At least one of the first or second jaw members is movable relative to the other from a spaced apart position to an approximated position to grasp tissue between the tissue contacting surfaces. The first and second jaw members are adapted to connect to a source of energy for electrosurgically treating tissue grasped between the tissue contacting surfaces. Each of the first and second thermal cutting elements is disposed on or within one of the first or second jaw members and adapted to connect to a source of energy for thermally treating tissue.

### CROSS-REFERENCE TO RELATED APPLICATIONS

This application is a 371 National Stage Application of International Application No. PCT/US2021/045540, filed Aug. 11, 2021, which claims benefit of U.S. Provisional Patent Application No. 63/072,840, filed Aug. 31, 2020, the entire contents of each of which is hereby incorporated herein by reference.

### FIELD

The present disclosure relates to electrosurgical instruments and, more particularly, to electrosurgical instruments including thermal cutting elements to facilitate tissue treatment, e.g., sealing, cutting, and/or coagulation of tissue.

### BACKGROUND

A surgical forceps is a pliers like instrument that relies on mechanical action between its jaw members to grasp, clamp, and constrict tissue. Electrosurgical forceps utilize both mechanical clamping action and energy to heat tissue to treat, e.g., coagulate, cauterize, or seal, tissue. Typically, once tissue is treated, the surgeon has to accurately sever the treated tissue. Accordingly, many electrosurgical forceps are designed to incorporate a knife that is advanced between the jaw members to cut the treated tissue. As an alternative to a mechanical knife, an energy based tissue cutting element may be provided to cut the treated tissue using energy, e.g., thermal, electrosurgical, ultrasonic, light, or other suitable energy.

### SUMMARY

As used herein, the term “distal” refers to the portion that is being described which is farther from an operator (whether a human surgeon or a surgical robotic), while the term “proximal” refers to the portion that is being described which is closer to the operator. Terms including “generally,” “about,” “substantially,” and the like, as utilized herein, are meant to encompass variations up to and including plus or minus 10 percent to take into account, for example, material, measurement, manufacturing, environmental, use, and/or other tolerances and variations. Further, to the extent consistent, any or all of the aspects detailed herein may be used in conjunction with any or all of the other aspects detailed herein.

Provided in accordance with aspects of the present disclosure is an electrosurgical instrument including an end effector assembly having first and second jaw members and at least first and second thermal cutting elements. Each of the first and second jaw members includes an electrically conductive tissue contacting surface. At least one of the first or second jaw members is movable relative to the other from a spaced apart position to an approximated position to grasp tissue between the tissue contacting surfaces. The first and second jaw members are adapted to connect to a source of energy for electrosurgically treating tissue grasped between the tissue contacting surfaces. Each of the first and second thermal cutting elements is disposed on or within one of the first or second jaw members and adapted to connect to a source of energy for thermally treating tissue.

In an aspect of the present disclosure, the first and second thermal cutting elements are arranged longitudinally such that, at least in the approximated position, the first thermal cutting element is at least partially more-proximal and such that the second thermal cutting element is at least partially more-distal.

In another aspect of the present disclosure, the first and second thermal cutting elements are disposed on the same jaw member. Alternatively, the first and second thermal cutting elements are disposed on different jaw members.

In still another aspect of the present disclosure, the first and second thermal cutting elements cooperate to extend substantially an entire length of a tissue treating zone defined between the electrically conductive tissue contacting surfaces in the approximated position.

In yet another aspect of the present disclosure, the first thermal cutting element is disposed on or within the first jaw member, the second thermal cutting element is disposed on or within the second jaw member, and in the approximated position, the first and second thermal cutting elements are at least partially aligned with one another.

In still yet another aspect of the present disclosure, the first and second thermal cutting elements are complementary to one another. Alternatively or additionally, the first and second thermal cutting elements define different widths.

In another aspect of the present disclosure, the first thermal cutting element is disposed on or within the first jaw member, the second thermal cutting element is disposed on or within the second jaw member, and in the approximated position, the first and second thermal cutting elements are at least partially offset relative to one another.

In yet another aspect of the present disclosure, a third thermal cutting element is disposed on or within one of the first or second jaw members.

In still another aspect of the present disclosure, the first thermal cutting element is disposed on or within the first jaw member and extends along at least a portion of the tissue contacting surface thereof and the second thermal cutting element is disposed on or within the second jaw member. The second thermal cutting element extends along at least a portion of the tissue contacting surface of the second jaw member and wraps at least partially around a distal tip of the second jaw member. The first and second thermal cutting elements are arranged longitudinally such that, at least in the approximated position, the first thermal cutting element is at least partially more-proximal and such that the second thermal cutting element is at least partially more-distal.

In another aspect of the present disclosure, the first thermal cutting element is disposed on or within the first jaw member and extends along at least a portion of the tissue contacting surface thereof and the second thermal cutting element is disposed on or within the first jaw member and extends along at least a portion of the tissue contacting surface thereof. The second thermal cutting element wraps at least partially around a distal tip of the first jaw member. The first and second thermal cutting elements are arranged longitudinally such that the first thermal cutting element is at least partially more-proximal and such that the second thermal cutting element is at least partially more-distal.

In still yet another aspect of the present disclosure, at least one of the first or second thermal cutting elements is a ferromagnetic cutting element. The ferromagnetic cutting element may be a ferromagnetic cutting wire that, in some configurations, may provide automatic Curie temperature control upon supply of energy thereto.

In yet another aspect of the present disclosure, the thermal cutting element includes a substrate including a heating layer disposed on at least a portion of the substrate. The substrate may be at least partially Plasma Electrolytic Oxidation (PEO) treated. The thermal cutting element may be configured to establish a thermal gradient to conduct heat from portions of the thermal cutting element not in contact with tissue to portions of the thermal cutting element in contact with tissue. Other suitable substrates are also contemplated such as, for example, ceramic, stainless steel, etc.

In still another aspect of the present disclosure, the electrosurgical instrument further includes a housing having a shaft extending distally therefrom. In such aspects, the end effector assembly may be disposed at a distal end of the shaft.

In another aspect of the present disclosure, the end effector is adapted to connect to an arm of a robotic surgical system.

### BRIEF DESCRIPTION OF DRAWINGS

The above and other aspects and features of the present disclosure will become more apparent in view of the following detailed description when taken in conjunction with the accompanying drawings wherein like reference numerals identify similar or identical elements.

FIG. 1 is a perspective view of a shaft based electrosurgical forceps provided in accordance with the present disclosure shown connected to an electrosurgical generator;

FIG. 2 is a perspective view of a hemostat style electrosurgical forceps provided in accordance with the present disclosure;

FIG. 3 is a schematic illustration of a robotic surgical instrument provided in accordance with the present disclosure;

FIG. 4A is an enlarged, side view of the end effector assembly of the shaft based electrosurgical forceps of FIG. 1 or otherwise configured for use with the hemostat style electrosurgical forceps of FIG. 2, the robotic surgical instrument of FIG. 3, or any other suitable surgical instrument, wherein the jaw members of the end effector assembly are disposed in a spaced apart position;

FIG. 4B is a side view of the end effector assembly of FIG. 4A, wherein the jaw members thereof are disposed in an approximated position;

FIG. 5 is a transverse, cross sectional view of the end effector assembly of FIG. 4A, wherein the jaw members thereof are disposed in the approximated position;

FIGS. 6-8 are transverse, cross sectional views of other end effector assemblies configured for use with the shaft based electrosurgical forceps of FIG. 1, the hemostat style electrosurgical forceps of FIG. 2, the robotic surgical instrument of FIG. 3, or any other suitable surgical instrument, wherein the jaw members of the respective end effector assemblies are disposed in the approximated position;

FIG. 9A is a side view of still another end effector assembly configured for use with the shaft based electrosurgical forceps of FIG. 1, the hemostat style electrosurgical forceps of FIG. 2, the robotic surgical instrument of FIG. 3, or any other suitable surgical instrument, wherein the jaw members thereof are disposed in a spaced apart position;

FIG. 9B is a side view of the end effector assembly of FIG. 9A, wherein the jaw members thereof are disposed in an approximated position;

FIGS. 10 and 11 are side views of j aw members of yet other end effector assemblies configured for use with the shaft based electrosurgical forceps of FIG. 1, the hemostat style electrosurgical forceps of FIG. 2, the robotic surgical instrument of FIG. 3, or any other suitable surgical instrument;

FIG. 12A is a side view of still yet another end effector assembly configured for use with the shaft based electrosurgical forceps of FIG. 1, the hemostat style electrosurgical forceps of FIG. 2, the robotic surgical instrument of FIG. 3, or any other suitable surgical instrument, wherein the jaw members thereof are disposed in a spaced apart position;

FIG. 12B is a side view of the end effector assembly of FIG. 12A, wherein the jaw members thereof are disposed in an approximated position;

FIG. 13 is a side view of another end effector assembly configured for use with the shaft based electrosurgical forceps of FIG. 1, the hemostat style electrosurgical forceps of FIG. 2, the robotic surgical instrument of FIG. 3, or any other suitable surgical instrument, wherein the jaw members thereof are disposed in a spaced apart position;

FIG. 14 is a transverse, cross sectional view of a thermal cutting element configured for use with some or all of the end effector assemblies of FIGS. 4A-13; and

FIG. 15 is a side view of another thermal cutting element configured for use with some or all of the end effector assemblies of FIGS. 4A-13.

### DETAILED DESCRIPTION

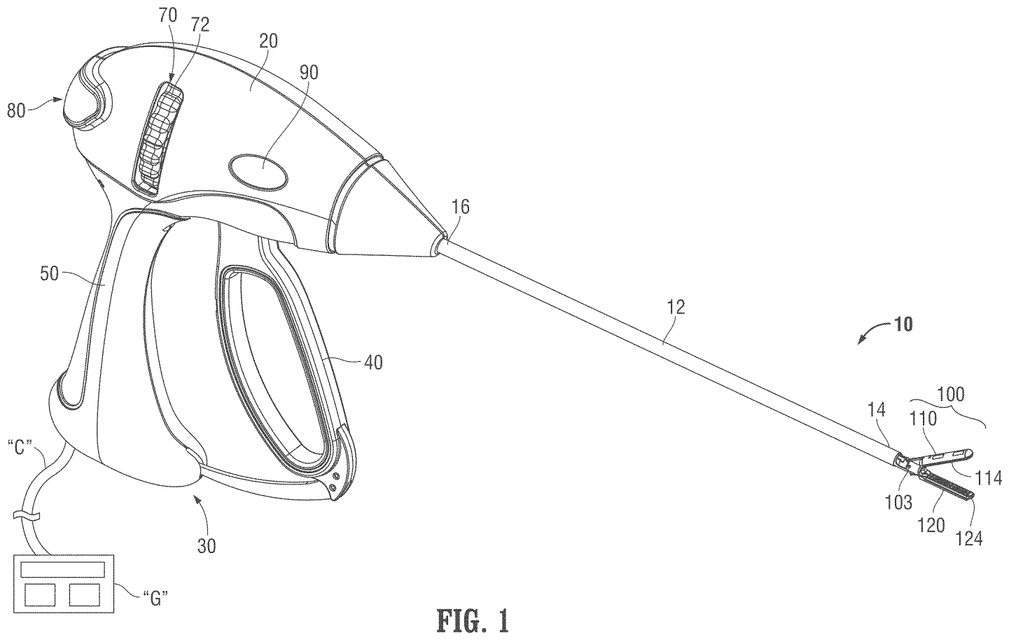

Referring to FIG. 1, a shaft based electrosurgical forceps provided in accordance with the present disclosure is shown generally identified by reference numeral 10. Aspects and features of forceps 10 not germane to the understanding of the present disclosure are omitted to avoid obscuring the aspects and features of the present disclosure in unnecessary detail.

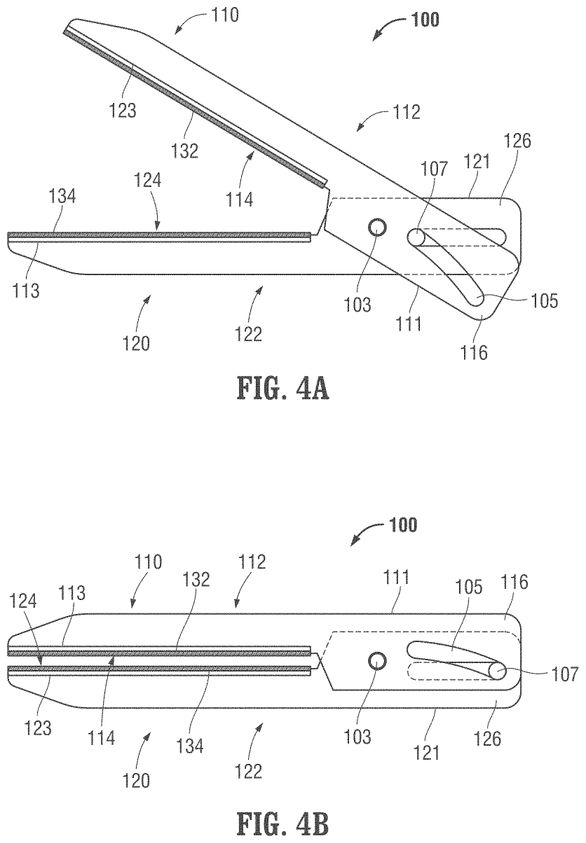

Forceps 10 includes a housing 20, a handle assembly 30, a rotating assembly 70, a first activation switch 80, a second activation switch 90, and an end effector assembly 100. Forceps 10 further includes a shaft 12 having a distal end portion 14 configured to (directly or indirectly) engage end effector assembly 100 and a proximal end portion 16 that (directly or indirectly) engages housing 20. Forceps 10 also includes cable “C” that connects forceps 10 to an energy source, e.g., a generator “G.” Cable “C” includes a wire (or wires) (not shown) extending therethrough that has sufficient length to extend through shaft 12 in order to connect to one or both tissue treating surfaces 114, 124 of jaw members 110, 120, respectively, and one or more thermal cutting elements 130 (see FIGS. 4A-13) of end effector assembly 100 to provide energy thereto. More specifically, first activation switch 80 is coupled to tissue treating surfaces 114, 124 (FIG. 4A) and the generator “G” for enabling the selective activation of the supply of energy to jaw members 110, 120 for electrosurgically treating, e.g., sealing, tissue. Second activation switch 90 is coupled to one or more thermal cutting elements 132, 134 (FIG. 4A) and the generator “G” for enabling the selective activation of the supply of energy to thermal cutting elements 132, 134 (FIG. 4A) and for thermally treating, e.g., cutting and/or coagulating, tissue. In configurations where multiple thermal cutting elements 132, 134 (FIG. 4A) are provided, activation switch 90 may be configured to collectively activate the thermal cutting elements 132, 134 (FIG. 4A), may include multiple activation positions each corresponding to one or more of the thermal cutting elements 132, 134 (FIG. 4A), may be associated with other switches, sensors, and/or feedback mechanisms to selectively activate one or more of the thermal cutting elements 132, 134 (FIG. 4A) based upon sensed parameters (instrument state (jaws open, jaws closed, etc.), tissue presence, whether sealing has been completed, etc.), and/or multiple second activation switches 90 may be provided for selectively activating the thermal cutting elements 132, 134 (FIG. 4A). Alternatively, a single activation switch, e.g., coupled to tissue treating surfaces 114, 124 (FIG. 4A), the one or more thermal cutting elements 132, 134 (FIG. 4A), and the generator “G” may be provided to initiate a seal and cut cycle via a single activation. Suitable feedback, sensor, or other suitable mechanism(s) to determine seal completion and to initiate cutting may be utilized.

Handle assembly 30 of forceps 10 includes a fixed handle 50 and a movable handle 40. Fixed handle 50 is integrally associated with housing 20 and handle 40 is movable relative to fixed handle 50. Movable handle 40 of handle assembly 30 is operably coupled to a drive assembly (not shown) that, together, mechanically cooperate to impart movement of one or both of jaw members 110, 120 of end effector assembly 100 about a pivot 103 between a spaced apart position and an approximated position to grasp tissue between tissue treating surfaces 114, 124 of jaw members 110, 120. As shown in FIG. 1, movable handle 40 is initially spaced apart from fixed handle 50 and, correspondingly, jaw members 110, 120 of end effector assembly 100 are disposed in the spaced apart position. Movable handle 40 is depressible from this initial position to a depressed position corresponding to the approximated position of jaw members 110, 120. Rotating assembly 70 includes a rotation wheel 72 that is selectively rotatable in either direction to correspondingly rotate end effector assembly 100 relative to housing 20.

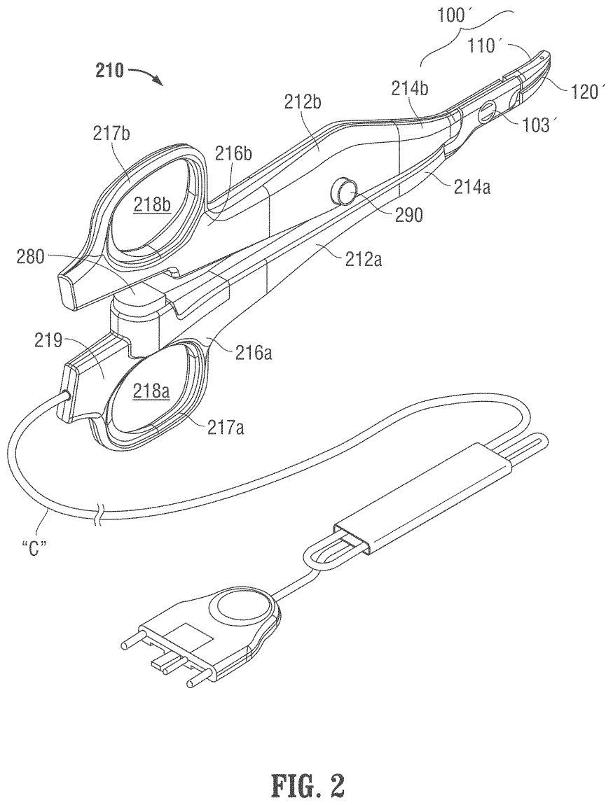

Referring to FIG. 2, a hemostat style electrosurgical forceps provided in accordance with the present disclosure is shown generally identified by reference numeral 210. Aspects and features of forceps 210 not germane to the understanding of the present disclosure are omitted to avoid obscuring the aspects and features of the present disclosure in unnecessary detail.

Forceps 210 includes two elongated shaft members 212a, 212b, each having a proximal end portion 216a, 216b, and a distal end portion 214a, 214b, respectively. Forceps 210 is configured for use with an end effector assembly 100′ similar to end effector assembly 100 (FIG. 4A). More specifically, end effector assembly 100′ includes first and second jaw members 110′, 120′ attached to respective distal end portions 214a, 214b of shaft members 212a, 212b. Jaw members 110′, 120′ are pivotably connected about a pivot 103′. Each shaft member 212a, 212b includes a handle 217a, 217b disposed at the proximal end portion 216a, 216b thereof. Each handle 217a, 217b defines a finger hole 218a, 218b therethrough for receiving a finger of the user. As can be appreciated, finger holes 218a, 218b facilitate movement of the shaft members 212a, 212b relative to one another to, in turn, pivot jaw members 110′, 120′ from the spaced apart position, wherein jaw members 110′, 120′ are disposed in spaced relation relative to one another, to the approximated position, wherein jaw members 110′, 120′ cooperate to grasp tissue therebetween.

One of the shaft members 212a, 212b of forceps 210, e.g., shaft member 212b, includes a proximal shaft connector 219 configured to connect forceps 210 to a source of energy, e.g., generator “G” (FIG. 1). Proximal shaft connector 219 secures a cable “C” to forceps 210 such that the user may selectively supply energy to jaw members 110′, 120′ and one or more thermal cutting elements 132, 134 (FIG. 4A) of end effector assembly 100′ for treating tissue. More specifically, a first activation switch 280 is provided for supplying energy to jaw members 110′, 120′ to electrosurgically treat tissue upon sufficient approximation of shaft members 212a, 212b, e.g., upon activation of first activation switch 280 via shaft member 212a. One or more second activation switch(es) 290 disposed on either or both of shaft members 212a, 212b is coupled to the thermal cutting element(s) 132, 134 (FIG. 4A) of end effector assembly 100′ and to generator “G” for enabling the selective activation of the supply of energy thereto for thermally treating tissue.

Jaw members 110′, 120′ define a curved configuration wherein each jaw member is similarly curved laterally off of a longitudinal axis of end effector assembly 100′. However, other suitable curved configurations including curvature towards one of the jaw members 110, 120′ (and thus away from the other), multiple curves with the same plane, and/or multiple curves within different planes are also contemplated. Jaw members 110, 120 of end effector assembly 100 (FIG. 1) may likewise be curved according to any of the configurations noted above or in any other suitable manner.

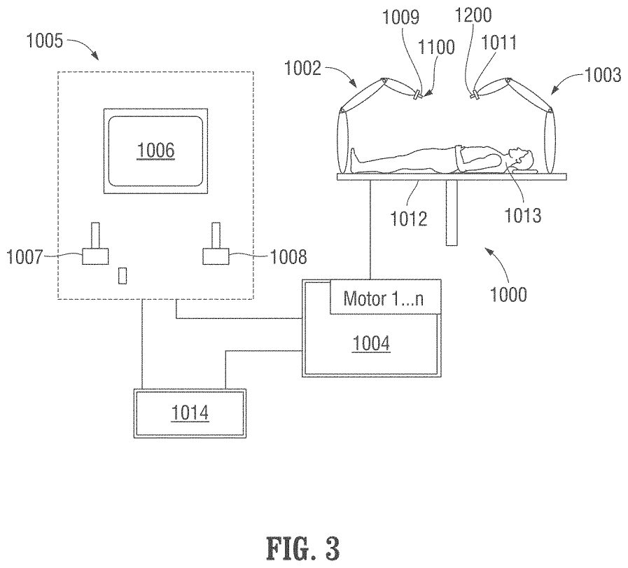

Referring to FIG. 3, a robotic surgical instrument provided in accordance with the present disclosure is shown generally identified by reference numeral 1000. Aspects and features of robotic surgical instrument 1000 not germane to the understanding of the present disclosure are omitted to avoid obscuring the aspects and features of the present disclosure in unnecessary detail.

Robotic surgical instrument 1000 includes a plurality of robot arms 1002, 1003; a control device 1004; and an operating console 1005 coupled with control device 1004. Operating console 1005 may include a display device 1006, which may be set up in particular to display three dimensional images; and manual input devices 1007, 1008, by means of which a surgeon may be able to telemanipulate robot arms 1002, 1003 in a first operating mode. Robotic surgical instrument 1000 may be configured for use on a patient 1013 lying on a patient table 1012 to be treated in a minimally invasive manner. Robotic surgical instrument 1000 may further include a database 1014, in particular coupled to control device 1004, in which are stored, for example, pre-operative data from patient 1013 and/or anatomical atlases.

Each of the robot arms 1002, 1003 may include a plurality of members, which are connected through joints, and an attaching device 1009, 1011, to which may be attached (directly or indirectly via intermediate structures, e.g., a housing and shaft extending from the housing), for example, an end effector assembly 1100, 1200, respectively. End effector assembly 1100 is similar to end effector assembly 100 (FIG. 1), although other suitable end effector assemblies for coupling to attaching device 1009 are also contemplated. End effector assembly 1200 may be any end effector assembly, e.g., an endoscopic camera, other surgical tool, etc. Robot arms 1002, 1003 and end effector assemblies 1100, 1200 may be driven by electric drives, e.g., motors, that are connected to control device 1004. Control device 1004 (e.g., a computer) may be configured to activate the motors, in particular by means of a computer program, in such a way that robot arms 1002, 1003, their attaching devices 1009, 1011, and end effector assemblies 1100, 1200 execute a desired movement and/or function according to a corresponding input from manual input devices 1007, 1008, respectively. Control device 1004 may also be configured in such a way that it regulates the movement of robot arms 1002, 1003 and/or of the motors.

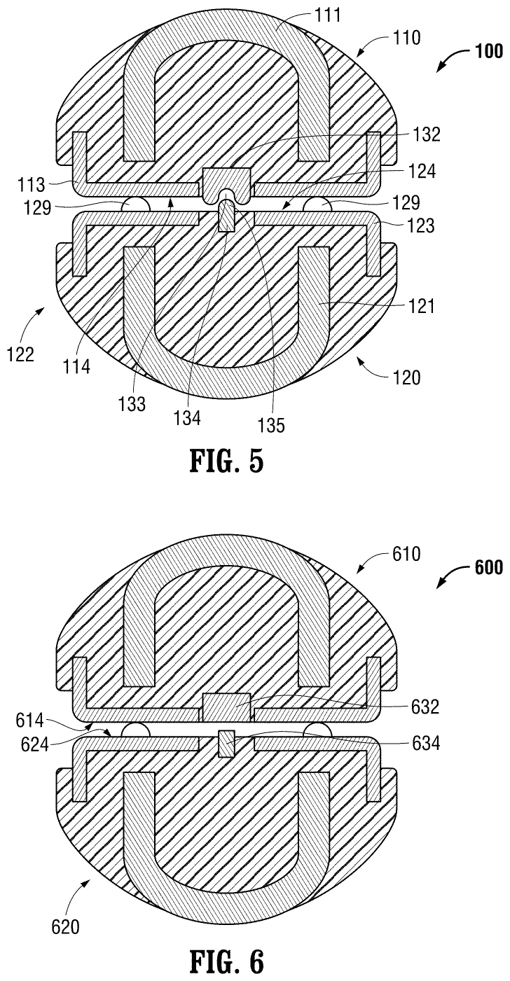

Turning to FIGS. 4A, 4B, and 5, end effector assembly 100, as noted above, includes first and second jaw members 110, 120. Each jaw member 110, 120 may include a structural frame 111, 121, a jaw body 112, 122, and a tissue treating plate 113, 123 defining the respective tissue treating surface 114, 124 thereof, although other configurations of either or both of jaw members 110, 120 (including different configurations for each jaw member 110, 120) are also contemplated.

Structural frames 111, 121 provide structural support to jaw members 110, 120 and include proximal flag portions 116, 126 that are operably coupled to one another and/or shaft 12 to enable pivoting of one or both of jaw members 110, 120 relative to the other (and shaft 12) between the spaced apart position and the approximated position for grasping tissue between tissue treating surfaces 114, 124. More specifically, proximal flag portions 116, 126 are pivotably coupled via a pivot 103 extending through aligned apertures defined therein. At least one of the proximal flag portions 116, 126 may further include a cam slot 105 that is configured to receive a cam pin 107 associated with the drive assembly (not shown) such that translation of a drive rod of the drive assembly (not shown), e.g., in response to actuation of movable handle 40 (FIG. 1) or a robotic drive, pivots jaw member 110 about pivot 103 and relative to jaw member 120 between the spaced apart position and the approximated position. However, other suitable drive arrangements and jaw movement configurations, e.g., bilateral jaws, are also contemplated.

Jaw bodies 112, 122 at least partially surround, support, and/or retain the components of the corresponding jaw members 110, 120, e.g., structural frames 111, 121 and tissue treating plates 113, 123. Jaw bodies 112, 122 may be formed from electrically and thermally insulative material(s) and may each be formed from a single, monolithic component or from multiple components. For example, jaw bodies 112, 122 may include outer jaw housings and inner jaw inserts, one or both of which is formed via overmolding onto jaw members 110, 120. Alternatively, some or all of the component(s) of jaw bodies 112, 122 may be separately formed and subsequently assembled with jaw members 110, 120.

In some configurations, tissue treating plates 113, 123 may be deposited onto jaw bodies 112, 122, e.g., via sputtering or other deposition techniques. Alternatively, tissue treating plates 113, 123 may be pre-formed and engaged with jaw bodies 112, 122 via, for example, overmolding, adhesion, mechanical engagement, etc. Lead wires (not shown) or other suitable electrical connecting structures are connected to tissue treating plates 113, 123 to connect tissue treating plates 113, 123 to generator “G” (FIG. 1) and/or first activation switch 80 (FIG. 1). More specifically, tissue treating plates 113, 123 are formed from electrically conductive material(s), e.g., for conducting RF electrosurgical energy therebetween for electrosurgically treating tissue, although tissue treating plates 113, 123 may alternatively be configured to conduct any other suitable energy, e.g., thermal, microwave, light, ultrasonic, etc., to or through tissue grasped therebetween for energy based tissue treatment. With respect to RF electrosurgical energy configurations, tissue treating plates 113, 123 may be energized to different potentials to establish a potential gradient for conducting energy through tissue disposed therebetween, e.g., to seal or otherwise electrosurgically treat the tissue. One or more stops 129 configured to inhibit shorting between tissue treating plates 113, 123 may be disposed on either or both tissue treating plates 113, 123.

Continuing with reference to FIGS. 4A, 4B, and 5, jaw members 110, 120, as noted above, includes structural frames 111, 121, jaw bodies 112, 122, and tissue treating plates 113, 123 defining tissue treating surfaces 114, 124, respectively. One or more thermal cutting elements 132, 134 are also provided such as, for example, a first thermal cutting element 132 disposed on or in jaw member 110 and a second thermal cutting element 134 disposed on or in jaw member 120.

Thermal cutting elements 132, 134, more specifically, extend longitudinally along tissue treating plates 113, 123 and may be disposed within longitudinally extending slots (see, e.g., FIG. 5) defined through tissue treating plates 113, 123 and/or portions of jaw bodies 112, 122. Alternatively, either or both thermal cutting elements 132, 134 may be disposed on respective tissue treating plates 113, 123 with or without an insulating layer(s) disposed therebetween. At least a portion of either or both of thermal cutting elements 132, 134 may be substantially co-planar with the corresponding tissue treating surface 114, 124, may be recessed relative thereto, and/or may protrude therefrom. As thermal cutting elements 132, 134 extend between opposing portions of respective tissue treating plate 113, 123, either or both thermal cutting elements 132, 134 may thus be utilized to cut sealed (or otherwise treated) tissue into two sealed tissue portions. Thermal cutting elements 132, 134 are selectively energizable (collectively or independently) to thermally treat, e.g., cut, tissue grasped between tissue treating plates 113, 123. Lead wires (not shown) or other suitable electrical connecting structures are connected to thermal cutting elements 132, 134 to connect thermal cutting elements 132, 134 to generator “G” (FIG. 1) and/or second activation switch(es) 90 (FIG. 1). Various different configurations of thermal cutting elements 132, 134 are detailed below with reference to FIGS. 5-8.

As shown in FIGS. 4A and 4B, thermal cutting elements 132, 134 extend along substantially entire lengths of respective tissue treating plates 113, 123. In this manner, thermal cutting elements 132, 134 extend substantially the entire length of the electrosurgical tissue treatment zone associated with jaw members 110, 120; that is, the length along which tissue may be electrosurgically treated, e.g., sealed, via tissue treating plates 113, 123, such that, for example, sealed tissue may be fully divided. However, as thermal cutting elements 132, 134 provide the ability to coagulate tissue in addition to cutting tissue, bleeding of tissue cut by thermal cutting elements 132, 134 outside the electrosurgical tissue treatment zone, e.g., tissue that has not been sealed, can be effectively controlled. Although detailed herein with respect to a length, it is also contemplated that the same apply to different widths, areas, heights, combinations thereof, etc.

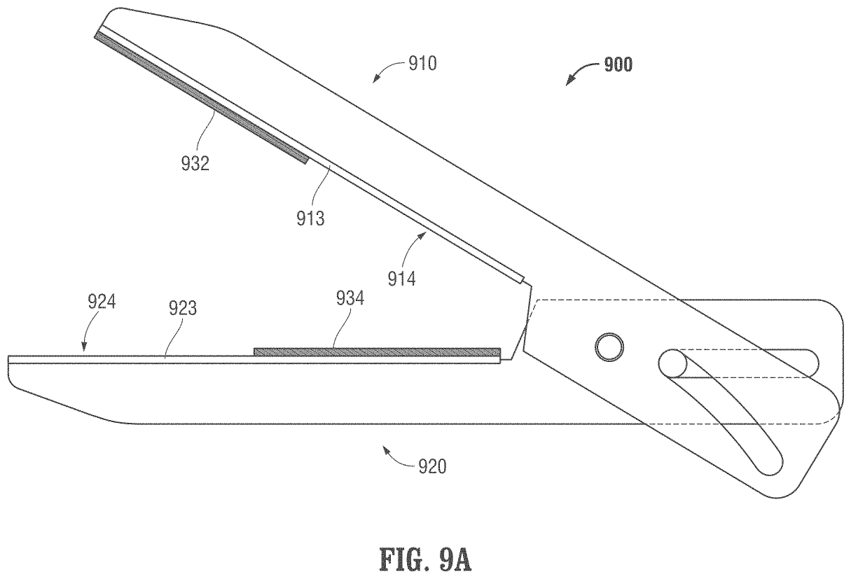

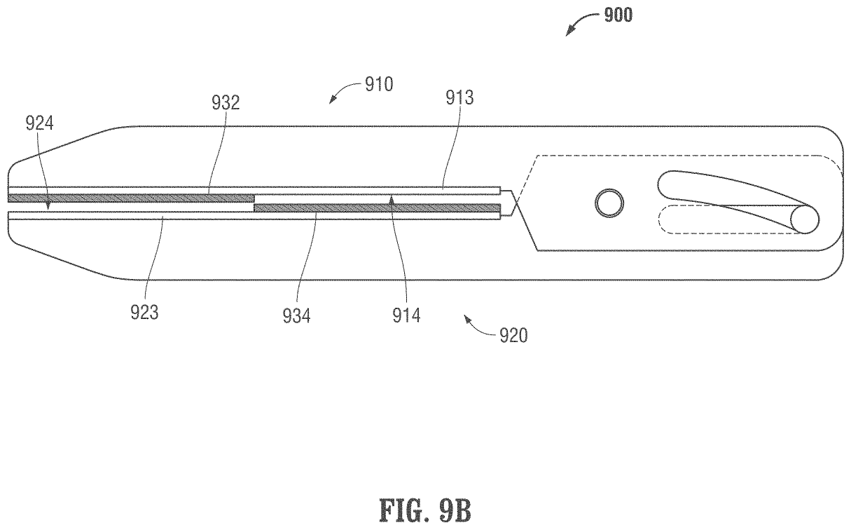

Referring to FIGS. 5-8 (and similarly applicable to FIGS. 9A-13), the thermal cutting elements may take various different forms and may be similar to one another or different from one another in size, shape, other configuration, type (ferromagnetic, resistive, etc.), number, etc. and/or may be positioned differently from one another. By providing differently configured and/or positioned thermal cutting elements, the thermal cutting elements may be used collectively (consecutively, simultaneously, in overlapping temporal relation, etc.) to facilitate a particular tissue treatment (e.g., cutting, coagulation, etc.) and/or individually for use in different situations or for different tissue treatments. For example, one mode using one or more of the thermal cutting elements may facilitate cutting grasped (and sealed) tissue while others modes using one or more thermal cutting elements may facilitate open jaw tissue cutting, cutting in a probe-like manner, coagulation, etc.

Thermal cutting elements 132, 134 illustrated in FIG. 5 define at least partially complementary configurations wherein one of the thermal cutting elements e.g., thermal cutting element 134 includes a protruding portion 135 and the other thermal cutting element, e.g., thermal cutting element 132, includes a recessed portion 133. Protruding portion 135 may remain spaced apart from recessed portion 133, may be configured for partial receipt therein, or may be configured to bottom out within recessed portion 133 to set a minimum gap distance between tissue treating surfaces 114, 124 in the approximated position of jaw members 110, 120 and/or to inhibit shorting therebetween. Alternatively or additionally, the one or more stops 129 may perform either or both of these functions. In embodiments where the one or more thermal cutting elements are disposed on the same jaw member or not aligned with one another, one or more of the thermal cutting elements may contacting the opposing tissue-treating surface to set the minimum gap distance. Any of the above-noted gap setting configurations may be utilized with any of the other configurations of end effector assemblies detailed herein.

FIG. 6 illustrates an end effector assembly 600 which may be similar to and include any of the features of end effector assembly 100 (FIGS. 4A-5), except that end effector assembly 600 includes thermal cutting elements 632, 634 disposed on or within jaw members 610, 620, respectively, that define different configurations relative to one another. More specifically, thermal cutting element 632 defines a first width while thermal cutting element 634 defines a second, smaller width. As an alternative or in addition to width variation, thermal cutting elements 632, 634 may define different heights (overall heights or heights relative to respective tissue contacting surfaces 614, 624 of jaw members 610, 620, respectively), different shapes, etc.

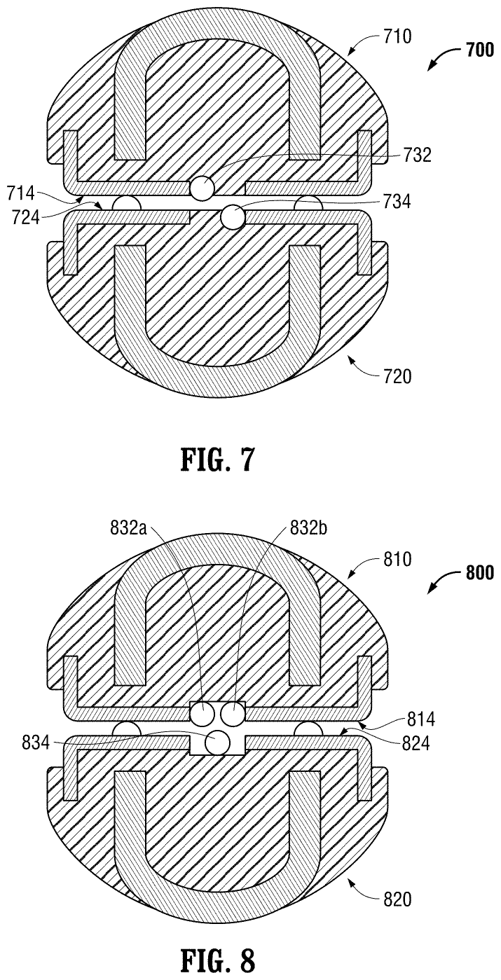

End effector assembly 700 illustrated in FIG. 7 may be similar to and include any of the features of end effector assembly 100 (FIGS. 4A-5), except that end effector assembly 700 includes thermal cutting elements 732, 734 disposed on or within jaw members 710, 720, respectively, and offset relative to one another. Thermal cutting elements 732, 734 may be laterally spaced apart from one another, laterally adjacent one another, or partially overlapping one another, and may define any suitable configurations similar or different from one another. Thermal cutting elements 732, 734 may oppose insulative portions of respective jaw members 720, 710 in the approximated position of jaw members 720, 710, or may oppose portions of respective tissue contacting surfaces 724, 714 in the approximated position of jaw members 720, 710.

FIG. 8 illustrates still another end effector assembly 800 similar to end effector assembly 100 (FIGS. 4A-5) and including any or all of the features thereof, except that end effector assembly 800 includes at least two thermal cutting elements 832a, 832b disposed on or within one of the jaw members, e.g., jaw member 810, and a different number of thermal cutting elements, e.g., one thermal cutting element 834, disposed on or within jaw member 820. Thermal cutting elements 832a, 832b may be laterally spaced apart from one another or laterally adjacent one another. Thermal cutting element 834 may be disposed laterally between (overlapping or adjacent) thermal cutting elements 832a, 832b, partially overlapping one or both of thermal cutting elements 832a, 832b, aligned with one of thermal cutting elements 832a, 832b, or offset relative to both thermal cutting elements 832a, 832b. Thermal cutting elements 832a, 832b, 834 may oppose one another in the approximated position of jaw members 810, 820, may oppose insulative portions of respective jaw members 820, 810 in the approximated position of jaw members 820, 810, and/or may oppose portions of respective tissue contacting surfaces 824, 814 in the approximated position of jaw members 820, 810.

Referring to FIGS. 9A and 9B, an end effector assembly 900 which may be similar to and include any of the features of end effector assembly 100 (FIGS. 4A-5) or any other end effector assembly detailed herein is shown including first and second jaw members 910, 920 each having a tissue treating plate 913, 923 defining a tissue treating surface 914, 924. A first thermal cutting element 932 is disposed on or in jaw member 910 and a second thermal cutting element 934 is disposed on or in jaw member 920.

Thermal cutting elements 932, 934, more specifically, extend longitudinally along portions (less than the entire lengths) of respective tissue treating plates 913, 923 and may be disposed within longitudinally extending slots defined through tissue treating plates 913, 923 or on tissue treating plates 913, 923 with or without an insulating layer(s) disposed therebetween. Although each thermal cutting elements 932, 934 extends less than the entire length of the corresponding tissue treating plate 913, 923, thermal cutting elements 932, 934 are arranged such that, in the approximated position of jaw members 910, 920, thermal cutting elements 932, 934 collectively extend along substantially the entire length of the electrosurgical tissue treatment zone associated with jaw members 910, 920; that is, the length along which tissue may be electrosurgically treated, e.g., sealed, via tissue treating plates 913, 923, such that, for example, sealed tissue may be fully divided. Thermal cutting elements 932, 934 may be disposed longitudinally adjacent one another, may at least partially overlap, or may define a relatively small longitudinal gap therebetween in the approximated position of jaw members 910, 920. Thermal cutting elements 932, 934 are selectively energizable (collectively or independently) to thermally treat, e.g., cut, tissue grasped between tissue treating plates 913, 923. Thermal cutting element 932 and/or thermal cutting element 934 may likewise be selectively energizable to treat tissue in a jaws open condition, e.g., via tenting.

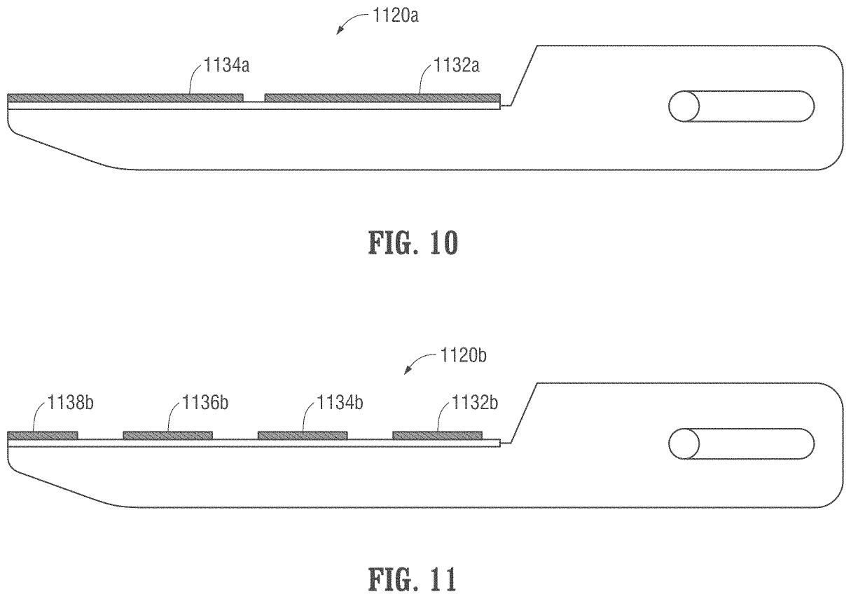

FIGS. 10 and 11 illustrate jaw members 1120a, 1120b of other end effector assemblies provided in accordance with the present disclosure which are similar to and may include any of the features of any of the other end effector assemblies detailed herein. Jaw members 1120a, 1120b, more specifically, may be configured for use with corresponding jaw members (not shown) of similar, complementary, or different configuration, with or without thermal cutting elements. Jaw members 1120a, 1120b includes two or more thermal cutting elements 1132a, 1134a and 1132b, 1134b, 1136b, 1138b that collectively extend along substantially the entire length of the respective tissue treating plate 1123a, 1123b of the respective jaw member 1120a, 1120b and may be disposed longitudinally adjacent one another, may at least partially overlap, or may define a relatively small longitudinal gap therebetween. Thermal cutting elements 1132a, 1134a of jaw member 1120a are selectively energizable (collectively or independently) to thermally treat, e.g., cut, grasped tissue; thermal cutting elements 1132b, 1134b, 1136b, 1138b of jaw member 1120b are likewise selectively energizable (collectively or independently) to thermally treat, e.g., cut, grasped tissue. Some or all of the thermal cutting elements 1132a, 1134a, 1132b, 1134b, 1136b, 1138b may likewise be selectively energizable to treat tissue in a jaws open condition, e.g., via tenting. As an alternative to separate multiple thermal cutting elements on one or both jaw members, each jaw member may include a single thermal cutting element having a plurality of different tissue-contacting portions (adjacent or spaced-apart from one another, e.g., defining a stepped configuration, serrated configuration, corrugated configuration (macro or micro), etc.

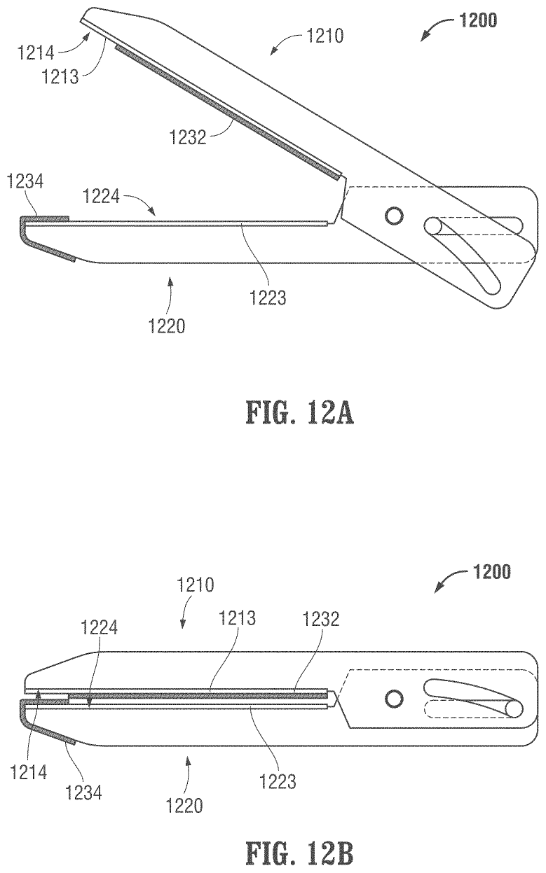

Turning to FIGS. 12A and 12B, another end effector assembly 1200 similar to and/or including any of the features of end effector assembly 100 (FIGS. 4A-5) or any other end effector assembly detailed herein is shown including first and second jaw members 1210, 1220 each having a tissue treating plate 1213, 1223 defining a tissue treating surface 1214, 1224. A first thermal cutting element 1232 is disposed on or in jaw member 1210 and a second thermal cutting element 1234 is disposed on or in jaw member 1220.

Thermal cutting elements 1232, 1234, more specifically, extend longitudinally along portions (less than the entire lengths) of respective tissue treating plates 1213, 1223 and may be disposed within longitudinally extending slots defined through tissue treating plates 1213, 1223 or on tissue treating plates 1213, 1223 with or without an insulating layer(s) disposed therebetween. Although each thermal cutting elements 1232, 1234 extends less than the entire length of the corresponding tissue treating plate 1213, 1223, thermal cutting elements 1232, 1234 are arranged such that, in the approximated position of jaw members 1210, 1220, thermal cutting elements 1232, 1234 collectively extend along substantially the entire length of the electrosurgical tissue treatment zone associated with jaw members 1210, 1220. Further, one of the thermal cutting elements, e.g., thermal cutting element 1234, extends distally beyond the electrosurgical tissue treatment zone and wraps at least partially around a distal tip of the corresponding jaw member, e.g., jaw member 1220. In some configurations, thermal cutting element 1232 extends along a greater length of the electrosurgical tissue treatment zone as compared to thermal cutting element 1234, e.g., defining a length ratio of at least, for example, 7:3, 8:2; or 9:1. Thermal cutting elements 1232, 1234 are selectively energizable (collectively or independently) to thermally treat, e.g., cut, tissue grasped between tissue treating plates 1213, 1223. Further, thermal cutting element 1234 (at least the wrapped portion thereof) may be utilized for dissection, spot coagulation, tunneling, enterotomies, back scoring, or other tissue treatment. As an alternative to length-wise orientation of thermal cutting elements 1232, 1234, other suitable configurations may be provided such as, for example, end-to-side arrangement, side-to-side arrangement, wrap-around arrangements, vertically-offset arrangements, horizontally-offset arrangements, etc.

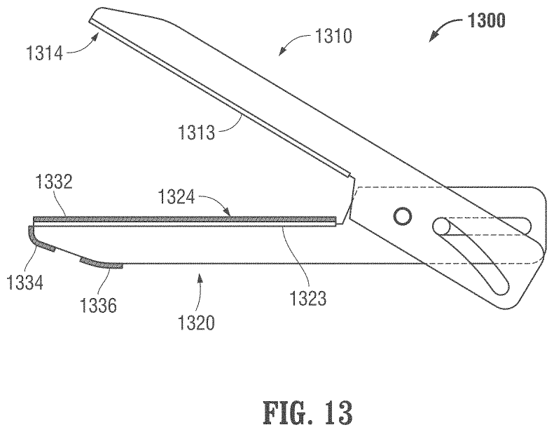

FIG. 13 illustrates another end effector assembly 1300 similar to and/or including any of the features of end effector assembly 100 (FIGS. 4A-5) or any other end effector assembly detailed herein. End effector assembly 1300 is shown including first and second jaw members 1310, 1320 each having a tissue treating plate 1313, 1323 defining a tissue treating surface 1314, 1324. First, second, and third thermal cutting elements 1332, 1334, 1336 are disposed on or in jaw member 1320, although additional or alternative thermal cutting elements may be disposed on or in jaw member 1310.

Thermal cutting element 1332 extends longitudinally along substantially the entire length of tissue treating plate 1323 and may be disposed within a longitudinally extending slot defined through tissue treating plate 1323 or on tissue treating plate 1323 with or without an insulating layer(s) disposed therebetween. Thermal cutting element 1334 wraps about at least a portion of a distal tip of jaw member 1320, and thermal cutting element 1336 extends about at least a portion of a backside of jaw member 1320. Thermal cutting elements 1332, 1334, 1336 are selectively energizable (collectively or independently) to thermally treat, e.g., cut, tissue grasped between tissue treating plates 1313, 1323. Further, thermal cutting elements 1334, 1336 may be utilized (collectively or independently) for dissection, spot coagulation, tunneling, enterotomies, back scoring, or other tissue treatment.

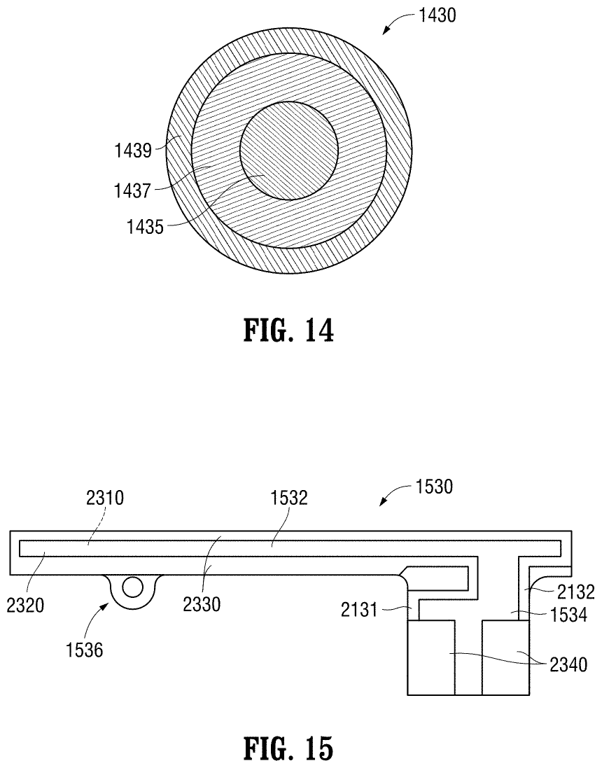

Turning to FIG. 14, some or all of the above detailed thermal cutting elements may be any suitable thermal cutting element such as, for example, a thermal cutting wire 1430 configured as a ferromagnetic thermal cutting wire including a solid conductive core 1435 and one or more layers of ferromagnetic coating 1437, 1439 disposed about solid conductive core 1435. More specifically, ferromagnetic thermal cutting wire 1430 includes solid conductive core 1435, e.g., copper, inner ferromagnetic coating 1437 disposed about solid conductive core 1435, and outer ferromagnetic coating 1439 disposed about inner ferromagnetic coating 1437. Inner and outer ferromagnetic coatings 1437, 1439 are formed from different materials and may define different thicknesses and/or overall volumes. Inner ferromagnetic coating 1437 may define a greater overall greater volume than outer ferromagnetic coating 1439 and/or may be formed from a relatively high magnetic loss material (as compared to outer ferromagnetic coating 1439) while outer ferromagnetic coating 1439 is formed from a material having a relatively higher permeability (as compared to inner ferromagnetic coating 1437). As a result of this configuration, current is more concentrated and generates high ohmic loss within outer ferromagnetic coating 1439 while the rest of the current within the relatively larger volume of the inner ferromagnetic coating 1437 generates more magnetic loss, e.g., hysteresis loss.

Thermal cutting wire 1430 is configured for self-limiting temperature regulation to achieve and maintain a pre-determined temperature. More specifically, in the presence of a high frequency alternating current, ferromagnetic materials generate large amounts of heat through the hysteresis of the magnetic field in the alternating current. Ferromagnetic materials also have a temperature where they cease to be ferromagnetic, referred to as the Curie temperature. Thus, once the material reaches the Curie temperature, the heating effect essentially ceases. That is, once the material ceases to be ferromagnetic, it becomes a much less effective heater thereby greatly decreasing its thermal output to the point where that temperature is maintained. Thus, the result is a heater that maintains a specific temperature based on its configuration and can be used to ensure sufficient heating and prevent overheating without the need for sensors, feedback mechanisms, and/or control loops. Further, in use, when the heated thermal cutting wire 1430 contacts tissue and is cooled below the Curie temperature, e.g., by virtue of contact with the relatively cooler tissue, the ferromagnetic thermal cutting wire 1430 again becomes ferromagnetic and once again becomes an effective heater to automatically heat back to the Curie temperature, thus providing self regulation. In other configurations, current may be increased to exceed the Curie temperature and to force ohmic heating on the thermal cutting wire 1430. In such configurations, the result is a drastic impedance change at the Curie point which can be utilized as feedback to control the temperature of the thermal cutting wire 1430.

Inner and outer ferromagnetic coatings 1437, 1439 may be configured to define different Curie temperatures. More specifically, outer ferromagnetic coating 1439 may define a Curie temperature that is less than the Curie temperature of inner ferromagnetic coating 1437. As a result of this configuration, when the Curie temperature of the outer ferromagnetic coating 1439 is first achieved, the output power does not immediately drop to zero (or close to zero); instead, the output power drops to a mid point of power due to the fact that the inner ferromagnetic coating 1437 maintains its magnetic properties and continues to be heated (via a lower output power) until it reaches its Curie temperature. The final temperature of thermal cutting wire 1430 in such embodiments is between the Curie temperature of outer ferromagnetic coating 1439 and the Curie temperature of inner ferromagnetic coating 1437, while the transition of output power (from the relatively high power when both coatings 1437, 1439 are being heated to the relatively lower output power when only inner coating 1437 is being heated) is relatively smooth.

Thermal cutting wire 1430 may further include an electrically insulative, e.g., ceramic, coating surrounding at least a portion of the outer ferromagnetic coating 1439. Thermal cutting wire 1430 may include a single exposed wire portion or multiple wire portions formed form multiple strands of wire or a single wire folded back onto, under, or along itself. Other configurations including non wire ferromagnetic cutting elements, are also contemplated.

With reference to FIG. 15, some or all of the above detailed thermal cutting elements may alternatively be configured as thermal cutting element(s) 1530 having an aluminum substrate at least a portion of which is Plasma Electrolytic Oxidation (PEO) treated with a heating layer affixed thereto such that when an AC voltage is applied, the thermal cutting element 1530 is heated for thermally cutting tissue in contact therewith. Other suitable substrates include ceramic, stainless steel, or any other metal capable of withstanding heating temperatures. Further, where the substrate is metallic, an insulative layer (or other suitable insulator) is provided to inhibit shorting. With respect to a stainless steel (and other) substrates, the insulator may be glass, e.g., sintered onto the surface of the substrate. In some configurations, thermal cutting element 1530 may include a substrate 2310, a PEO coating 2320 disposed about substrate 2310, a heating layer 2330 disposed on the PEO coating 2320 to form a heating element circuit including first and second end portions 2331, 2332, and first and second contacts 2340 electrically coupled to the respective first and second end portions 2331, 2332 of heating layer 2330. Thermal cutting element 1530 defines an elongated body 1532, a proximal connection flange 1534 extending from a proximal end portion of elongated body 1532, and one or more attachment flanges 1536 extending from, e.g., a central or distal end portion elongated body 1532, to facilitate attachment to or within a jaw member. First and second end portions 2331, 2332 of heating layer 2330 are disposed at proximal connection flange 1534. Heating layer 2330 defines a continuous circuit trace including first and second spaced apart segments extending from first and second end portions 2331, 2332, respectively, distally along elongated body 1532 to or adjacent a distal end portion of elongated body 1532, wherein the first and second segments are interconnected with one another via a connector segment of heating layer 2330.

First and second contacts 2340 are affixed to first and second end portions 2331, 2332, respectively, of heating layer 2330 at proximal connection flange 1534 to enable connection of electrical lead wires thereto for applying an AC voltage thereto to heat thermal cutting element 1530. Although shown in one orientation and position in FIG. 15, other suitable orientations, positions, and/or other configurations of contacts 2340 are also contemplated. Proximal connection flange 1534 may extend orthogonally relative to a longitudinal axis of elongated body 1532, although other configurations are also contemplated.

In use, with tissue in contact with thermal cutting element 1530 and an AC voltage applied (although, in other configurations a DC voltage may be used) across first and second contacts 2340 to thereby heat thermal cutting element 230, a thermal gradient is established whereby, as a result of the configuration of thermal cutting element 1530, heat is conducted from portions of thermal cutting element 1530 not in contact with tissue to those portions that are in contact with tissue. This facilitates heating and controlling the temperature of the portion of thermal cutting element 1530, e.g., the body portion or the distal probe portion, that is in contact with tissue, thus facilitating control of the cutting (or other thermal treatment) of tissue.

Other suitable thermal cutting elements are also contemplated.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. An electrosurgical instrument, comprising:
an end effector assembly, including:
first and second jaw members each including an electrically conductive tissue contacting surface, at least one of the first or second jaw members movable relative to the other from a spaced apart position to an approximated position to grasp tissue between the tissue contacting surfaces, the first and second jaw members adapted to connect to a source of energy for electrosurgically treating tissue grasped between the tissue contacting surfaces; and
at least first and second thermal cutting elements, each of the first and second thermal cutting elements disposed on or within one of the first or second jaw members and adapted to connect to a source of energy for thermally treating tissue.

2. The electrosurgical instrument according to claim 1, wherein the first and second thermal cutting elements are arranged longitudinally such that, at least in the approximated position, the first thermal cutting element is at least partially more-proximal and such that the second thermal cutting element is at least partially more-distal.

3. The electrosurgical instrument according to claim 2, wherein the first and second thermal cutting elements are disposed on the same jaw member.

4. The electrosurgical instrument according to claim 2, wherein the first and second thermal cutting elements are disposed on different jaw members.

5. The electrosurgical instrument according to claim 2, wherein the first and second thermal cutting elements cooperate to extend substantially an entire length of a tissue treating zone defined between the electrically conductive tissue contacting surfaces in the approximated position.

6. The electrosurgical instrument according to claim 1, wherein the first thermal cutting element is disposed on or within the first jaw member, the second thermal cutting element is disposed on or within the second jaw member, and in the approximated position, the first and second thermal cutting elements are at least partially aligned with one another.

7. The electrosurgical instrument according to claim 6, wherein the first and second thermal cutting elements are complementary to one another.

8. The electrosurgical instrument according to claim 6, wherein the first and second thermal cutting elements define different widths.

9. The electrosurgical instrument according to claim 1, wherein the first thermal cutting element is disposed on or within the first jaw member, the second thermal cutting element is disposed on or within the second jaw member, and in the approximated position, the first and second thermal cutting elements are at least partially offset relative to one another.

10. The electrosurgical instrument according to claim 9, further comprising a third thermal cutting element disposed on or within one of the first or second jaw members.

11. The electrosurgical instrument according to claim 1, wherein the first thermal cutting element is disposed on or within the first jaw member and extends along at least a portion of the tissue contacting surface thereof, the second thermal cutting element is disposed on or within the second jaw member and extending along at least a portion of the tissue contacting surface thereof and wrapping at least partially around a distal tip of the second jaw member, the first and second thermal cutting elements arranged longitudinally such that, at least in the approximated position, the first thermal cutting element is at least partially more-proximal and such that the second thermal cutting element is at least partially more-distal.

12. The electrosurgical instrument according to claim 1, wherein the first thermal cutting element is disposed on or within the first jaw member and extends along at least a portion of the tissue contacting surface thereof, the second thermal cutting element is disposed on or within the first jaw member and extending along at least a portion of the tissue contacting surface thereof and wrapping at least partially around a distal tip of the first jaw member, the first and second thermal cutting elements arranged longitudinally such that the first thermal cutting element is at least partially more-proximal and such that the second thermal cutting element is at least partially more-distal.

13. The electrosurgical instrument according to claim 1, wherein at least one of the first or second thermal cutting elements is a ferromagnetic cutting element.

14. The electrosurgical instrument according to claim 13, wherein the ferromagnetic cutting element is a ferromagnetic cutting wire.

15. The electrosurgical instrument according to claim 13, wherein the ferromagnetic cutting element provides automatic Curie temperature control upon supply of energy thereto.

16. The electrosurgical instrument according to claim 1, wherein the thermal cutting element includes a substrate including a heating layer disposed on at least a portion of the substrate.

17. The electrosurgical instrument according to claim 16, wherein the substrate is at least partially Plasma Electrolytic Oxidation (PEO) treated.

18. The electrosurgical instrument according to claim 16, wherein thermal cutting element is configured to establish a thermal gradient to conduct heat from portions of the thermal cutting element not in contact with tissue to portions of the thermal cutting element in contact with tissue.

19. The electrosurgical instrument according to claim 1, further comprising:
a housing having a shaft extending distally therefrom, wherein the end effector assembly is disposed at a distal end of the shaft.

20. The electrosurgical instrument according to claim 1, wherein the end effector is adapted to connect to an arm of a robotic surgical system.
